(19) United States
(12) Patent Application Publication
(10) Pub. No.: US 2010/0065064 A1
(43) Pub. Date: Mar. 18, 2010

(54) Condom Applicator Device

(76) Inventor: Mor Maty Seck, Yaounde (CM)

Correspondence Address: NT Patent Law, 6039 E. Grant Rd., Tucson, AZ, 85712, US

(21) Appl. No.: 12/296,094

(22) PCT Filed: Apr. 4, 2007
(86) PCT No.: PCT/OA2007/000001

§ 371 Date: Oct. 3, 2008

(30) Foreign Application Priority Data
Apr. 5, 2006 (OA) 1200600116

(51) Int. Cl. A61F 6/04

(52) U.S. Cl. 128/844

(57) ABSTRACT

Novel condom applicator device composed of a collar (3) with a projection at its end, the inner diameter of which rests on top of the penis, a flat ring (1) in which the operation for applying the condom (P) is performed by means of a lining (6) combined with its outer diameter. The device is symmetrically separated along its vertical axis. It is joined by means of the lining (6) at the level of the notch (e1) or by means of the collar (3) at the level of the notch (e2). A cap (7) stabilizes its two ends (a1) and (a2). The notch (e) is automatically separable whether a rigid material is used, or whether the device is flexible in order to accommodate a means for preserving same with the condom placed in the center thereof in a special wrapper.

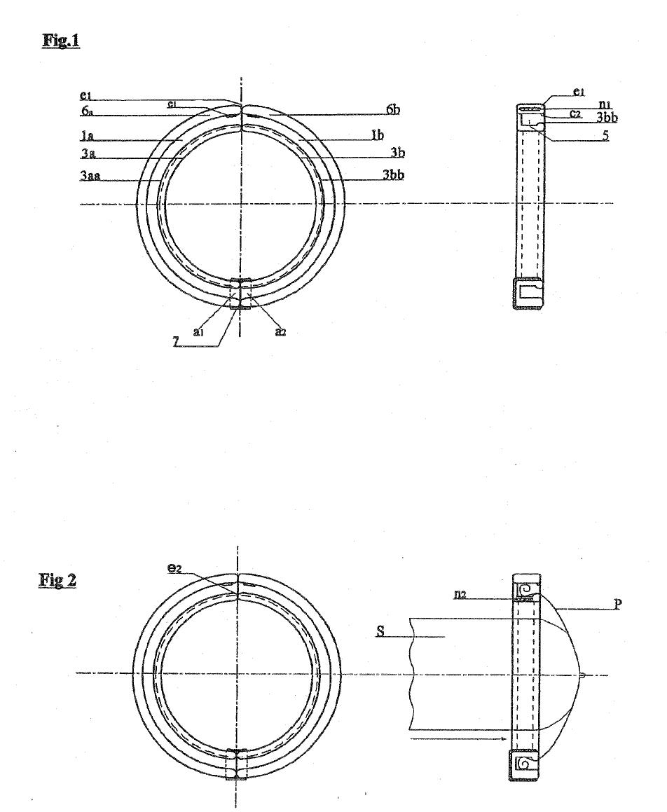

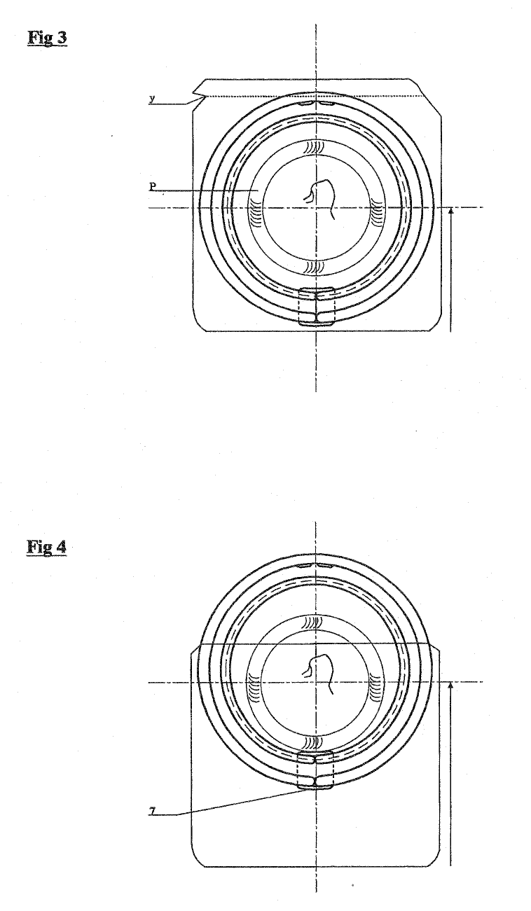

This invention is a device that helps to insert a male's sexual organ into a condom with ease and accuracy.

The novel condom applicator device consists of an open hollow ring on which the condom is mounted, the ring being symmetrically separable thanks to a notch.

The notch is automatically separable, whether the device material is fragile or flexible deprived of the notch in order to preserve it with the condom placed in its middle in a special wrapper.

The manual method for placing the condom on the penis brings with it a few practical problems.

Indeed, the condom is specific in that there is a flexible unfolding string of a smaller diameter than a penis on erection. That is why the latter's manual unfolding may prove difficult, with creasing of the penis skin that may result in the condom being ejected from the sex, in its degradation (air infiltration). In addition to that, although flexible, pinching the condom's end with one hand while unfolding it with the other is, in practice, a difficult exercise. Men and notably those of them who perform in darkness may err about the normal way of placing the condom. The sexual partners, often doubtful about the perfect placing of the condom by the user, may experience serious psychological difficulties.

It indeed takes a perfect placing of the condom by the user to ensure a better protection from STIs, and notably HIV/AIDS. Besides, some men and their partners would rather not use the condom because of the thickness of its membrane that robs them of the expected sensitiveness. Prior to this invention, Patent W02006/025755A3 has the same characteristics and functionalities which may:
Stabilise the condom membrane's unfolding string in a width position on top of the penis diameter for a quick, accurate and easy intromission to its bottom.
Ensure easiness of handling and gripping
Use a material that abides by the health standards and allows for its good functioning without any physical or chemical alteration of the condom.

In the event of its use outside the condom wrapper, the new device we have invented also offers some characteristics that may irremediably result in its destruction to deter any secondary use so as to comply with some specific standards of hygiene. It again offers, in the event of its being wrapped in a condom, a conservation procedure in a special wrapper for efficient long-lasting warehousing and stocking. Besides these objectives, the new invention has to allow for an easy and comfortable unfolding of ultra-fine membrane condoms.

It consists, according to the first version of Table 1, and for version 2 of Table 2, of a collar with a projection at its top (3) that rests in the inner diameter, higher than that of a penis (S) on erection, of a flat ring (1) in which the condom (P) mounting operation by means of a lining (6) occurs fully combined with its outer diameter. The device appears as a hollow ring which is symmetrically separated along its vertical axis into two hollow half-rings consisting of:
Half a collar (3a), half a flat ring (1a) and half a lining (6a) on the one hand
On the other, half a collar (3b), half a flat ring (1b) and half a lining (6b)
   As with Table 1, the two hollow half-rings are joined by means of the lining (6) at the level of the notch (e1) around its thickness with round angles located at the top of its vertical axis. This notch (e1) leaves a fine thickness (n1) variable according to the fragility of the material used, so as to provoke its easy breaking.

According to Table 2, the two hollow half-rings are joined through the collar at the level of its flat section (5), instead of the notch (e2) around this flat section with round angles located at the top of its vertical axis. This notch (e2) leaves a fine thickness (n2) variable according to the fragility of the material used to provoke its easy breaking.

Also, the material used has to have properties/components so as to allow for the thickness (n) of the breaking not any projection or pieces.

We then have two half-collars (3a) and (3b) that have at their respective tops, some round projection in the form of strings (3aa) and (3bb) which are used as 0.5 mm high support for the condom which nestles into the flat section (5) of a width slightly higher or equal to the width of the condom membrane's unfolding string in an enlarged position in the device.

We have two flat half-rings (1a) and (1b) of a width slightly higher or equal to the condom's unfolding string in an enlarged position in the device.

We have two half-linings (6a) and (6b) that make two half-circles of optional thickness and with the same height as the collar.

On either sides of the vertical axis of the lining (6) inner wall's top are fine projections in the form of small strings of small lengths and a 0.5-mm thickness at the same height as the support strings (3aa) and (3bb). These strings (c1) and (c2)'s role consists in offering strong maintenance to a portion of the condom membrane's folding string to make its full placement into the collar easier.

A global view of the device then gives two round-angled hollow half-rings which are either joined at the level of the device lining (6) or collar (3).

The device has two free round-angled (a1) and (a2) ends separated at the lower level of the vertical axis. The very fine thickness (n) does not guarantee balance for the two hollow half-rings, which is why the cap stabilises the (a1) and (a2) ends.

The cap (7), symmetrically set in relation to the vertical axis, has an optional thickness and a cavity of the same form as the free ends' other side (a1) and (a2). But the cap (7) is interdependent with the (a2) end and stabilises the (a1) end which strongly sinks into its cavity while having the possibility to be set over and stray away from the (a2) end.

The cap cavity in which the (a1) end nestles must be 3 mm long so as to deprive the user of the possibility to strongly reset the (a1) end after the lining or collar breaking in view of a secondary use of the device.

The materials used that bring in fragility such as Perspex and crystal PS may, thanks to the thickness' (n) great fineness, make the lining or collar break before any handling of the (a1) section so as to pull out the rest of the condom string by setting it over or by putting it away from the (a2) section. These materials then leave an (n1) or (n2) thickness slightly lower than 0.5 mm.

Strong materials such as PE or PP, polyolefines have some tenderness that constitutes a delaying factor for the notch breaking. They may however have a highly fine thickness to allow it to be torn.

According to Versions 1 and 2, mounting and pulling on the condom happen in the following way:
1) Insert with your two thumbs a section of the condom string into the device placed in a direction for slipping on.

The operation must be done in the section of the device lining with the two strings (c1) and (c2). By inserting a section of the condom string at this level, the string, because of its flexibility will shrink and then resume its initial shape inside the lining.
2) That way, the condom string strongly held may be easily and totally slipped into the collar again with pressure from the two thumbs.

During the operation, the breaking at the level of the thickness (n1) or (n2) notch (e) is avoided despite its fragility thanks to the cap (7) which maintains the (a1) and (a2) ends.

Pulling on the condom follows the stages below:
1—at the time of the penis (S) erection, hold the device with a hand and pinch the condom (P) end with the other while it is on erection and placed in the centre of the hole.
2—Place the condom into the penis, a light progressive push on the device unfolds the condom perfectly even if the erection is not hard enough. The very polished state of the collar surface where the condom string is placed as well as its support (3aa) and (3bb) make this operation possible. That's why the breaking at the level of the thickness (n2) notch in Table 2 offers the advantage of destroying Section 5's surface state to prevent any secondary unfolding of the condom.

Any attempt to discard the end (a1) or to take it out of the cap (7) will provoke a crack or make the thickness (n2) break even if sticking has been performed on the other side of the notch (e2) or on the lining (6). The device hole's superiority in relation to the penis (P) allows for a comfortable course up to the pubis bottom, however fine the condom string may be.

It's worth noting that during this operation, the (a1) extremity is maintained long enough in the cap (7) to mainly resist the penis push into the condom. The cap (7) has a cavity as wide as or slightly less wide than the free end's (a1) other side.
3.—The lining (6) also aims at protecting the condom string from the user's fingers so that it will be impossible for him to touch it or take it out of the device because of the short distance between the lining and the collar top.

The fine thickness (n) left by the notch, associated with the cap (7)'s small length, allows for the device's automatic division through a mere gesture, even during the operator's intercourse, who maintains it at his penis bottom after slipping on the condom.

One avoids, on the one hand in such a hypothesis to keep the device and on the other hand to cause it to re-emerge entirely, at the risk of seeing its inner wall, which would have been in contact with the penis during pulling on, to touch the membrane of the already worn condom.

After that, the user has no other resort but to perform a slight handling aiming at setting over the (a1) end or at discarding it from the (a2) end. Such a gesture is enough to cause the thickness (n1) or (n2) to break at the notch (e). Then, because of its flexibility, the rest of the condom string which tends to stick to the penis slips out of the device's two half-collars. The device destroys itself that way and can no longer be used a second time.

According to the applicator's version 3 Tables 3 and 4, the device must be manufactured with a flexible material that complies with the health standards and allowing for its good functioning without any physical or chemical alteration of the condom it goes with in a package.

Its easy accessibility (a condom, an applicator) might deter its second use even if it is deprived of the notch (e).

It offers the same functionalities and characteristics as Version 1. It comes as with Table 3 in a flexible material making a uniform hollow with a unique separation on the vertical axis at the level of the (a1) and (a2) ends. The ends are stabilised by the hat (7).

According to Table 3, the condom's conservation occurs in the following way:
1) Place the condom in the centre of the applicator's hole, its pulling on direction materialised by its end directed upwards depending on the device hollow.
2) Insert the device equipped with the condom in the wrapper. The condom's isolated position in the centre of the device helps to reduce the interaction effects between the two materials during their stocking.

Also, the device's tenderness and height protect the condom.

The package used is a special wrapper that has on its side a single trapeze-shaped notch (y) 1 mm down the device top.

This notch is for creating an opening signalled by a pre-drawn track on the two sides of the wrapper parallel to the device's horizontal axis 2 to 3 mm lower than the applicator's outer diameter.

Pulling on happens in the following way:
1. At the time of erection, tearing delicately the end of the wrapper using the notch (y). Part of the device appears thanks to the position of the notch (y)
2. Push the device out of the wrapper which stalls on the two ends at its opening because of its big outer diameter. Thanks to the device's inner diameter wall, this operation makes a section of the condom string slip out of the wrapper as shown by Table 4.
3. Seize the string end to insert it in the device's hollow without risking to err in the pulling on direction.

This operation is achievable in dark areas as locating is possible with the user touching the device's hollow after his seizing a section of the condom string.
4. A portion of the folding string being therefore in support of the device, the user shall pull out the remaining device from the wrapper thanks to its flexibility.
4.—Then set up the condom completely in the device. The condom consisting of a flexible, circular and uniform unfolding string, the device made of a hollow and uniform ring, too may contain it in an enlarged position in its collar even if it's flexible.
5.—Carry out its pulling on as indicated by versions 1 and 2
6.—After unfolding the condom to the penis bottom, dislodge the hat's (7) free end (a1) before withdrawing the whole device by twisting and then lifting that end, so that the remaining device follows suit because of its flexibility.

1. A new condom applicator device consisting on a collar (3) that rests at the level of the inner diameter of a flat ring (1) on top of a penis in which the condom (P) mounting operation occurs thanks to a lining (6) combined to its outer diameter. The device separated symmetrically on its vertical axis, it's joined by means of a lining (6) at the level of the notch (e1) or by means of the collar at the level of the notch (e2), and by means of its two ends (a1) and (a2) stabilized by the cap (7) or whether the device is flexible, deprived of the notch in order to accommodate a means for preserving same with the condom placed in the centre thereof in a special wrapper.

2-10. (canceled)
